(19) United States
(12) Patent Application Publication
(10) Pub. No.: US 2026/0195040 A1
(43) Pub. Date: Jul. 9, 2026

(54) SIMPLIFIED USER INTERFACE FOR MEDICAL INFUSION DEVICE

(71) Applicant: mylife Diabetes Care AG, Burgdorf (CH)

(72) Inventors: Lukas Gertsch, Solothurn (CH); Thomas Weinhold, Bern (CH)

(21) Appl. No.: 19/558,960

(22) Filed: Mar. 6, 2026

Related U.S. Application Data
(63) Continuation of application No. PCT/EP2024/072837, filed on Aug. 14, 2024.

(30) Foreign Application Priority Data
Sep. 12, 2023 (EP) 23196942.9

(51) Int. Cl. G06F 3/04886; A61M 5/172; G06F 3/01; G06F 3/041; G06F 3/0482; G06F 3/04845; G06F 3/16

(52) U.S. Cl. CPC G06F 3/04886; A61M 5/172; G06F 3/017; G06F 3/04166; G06F 3/04845; G06F 3/167; A61M 2205/505; G06F 3/0482; G06F 2203/04803

(57) ABSTRACT

A portable medical infusion pump includes a user interface with at least a touch sensitive display and a button and an electronic controlling assembly for operating and controlling the portable medical infusion pump configured to transition the user interface from a locked state to an unlocked state through an unlock process performed by a using person. The unlock process prevents inadvertent operation of the portable medical infusion pump.

### CROSS-REFERENCE TO RELATED APPLICATIONS

This application is a continuation of International Patent Application No. PCT/EP2024/072837, filed Aug. 14, 2024, entitled “SIMPLIFIED USER INTERFACE FOR MEDICAL INFUSION DEVICE,” which in turn claims priority to European Patent Application No. 23196942.9, filed Sep. 12, 2023, entitled “SIMPLIFIED USER INTERFACE FOR MEDICAL INFUSION DEVICE”, each of which is incorporated by reference herein, in its entirety and for all purposes.

### TECHNICAL FIELD

Implementations relate to user interfaces of portable medical infusion pumps.

### BACKGROUND

Portable ambulatory medical devices have proved useful for treating patients with medical conditions that require continuous monitoring and/or treatment. One example of such a portable ambulatory medical device is a device that involves the delivery of fluids. There are many applications in academic, industrial, and medical fields, as well as others, that involve devices capable of accurately and controllably delivering fluids, including liquids and gases, that have a beneficial effect when administered in known and controlled quantities. This is particularly true in the medical field, where treatments for many patients include the administration of a known amount of a substance at predetermined intervals. For example, the treatment of diabetes involves just such a regimented dosage of medicaments such as insulin. In addition, diabetes is one medical indication wherein patients routinely administer the medicament to themselves by a subcutaneous modality, such as a hypodermic syringe injection or by an ambulatory infusion pump. As such, providing a patient with the means to safely, reliably, and comfortably administer required doses of medication such as, e.g., insulin, may be particularly important in order to facilitate patient compliance and accurate treatment of the condition.

Some ambulatory medical devices include a touchscreen on which symbols may be displayed and from which inputs may be received for operation of the device. Other input mechanisms involve keyboards or hardware switches. In general, a series of display screens or windows are shown on a device display or on the device touchscreen, showing alphanumeric text and symbols, thereby providing menu screens through which the user can control operation of the device. User interaction, such as by touching the alphanumeric text and symbols, provides user input and facilitates navigation through the menu screens and selection and controlling the device functions.

The phenomenon of unintended, inadvertent activation of portable devices is not an uncommon occurrence. Telephone calls accidentally placed via a mobile telephone through inadvertent activation have become a fact of modern life. Such accidental calls can be annoying and troublesome for a mobile telephone user. In the case of a portable ambulatory medical device, such accidental activation can have serious consequences. In fact, in the case of portable ambulatory medical devices, any changes at all that are unintended or inadvertent may be problematic and even dangerous. For example, an untimely delivery of insulin, or delivery of an unexpectedly changed amount of insulin, or the absence of an expected dose, can have extremely deleterious results, and may even be dangerous to the user. User safety (and also usability) would be improved with a reduction in the likelihood of an accidental or unintended activation or deactivation of a portable ambulatory medical device.

US2020012401 A1 discloses an infusion pump with a touch screen. In order to prevent inadvertent operation of the pump the touch screen has a locked and an unlocked state. The pump comprises a processor which is configured to put the touch screen into the locked state which limits input received into the touch screen. It is further configured to display an unlock screen on the touch screen and subsequently to unlock the touch screen from the locked state to the unlocked state upon receiving a predetermined activation sequence comprising a sequence of predetermined alphanumeric characters selected through the touch screen. Activation sequence means that the displayed alphanumeric characters have to be touched in the correct sequence in order to make the processor changing the state. Illiterate people and younger children may be confused by the displayed alphanumerical characters on the unlock screen and may therefore not be able to unlock the touch screen.

Hence there is a need for simple user interfaces of infusion pumps, in particular in providing simplified but safe methods to firstly prevent inadvertent operation of the infusion pump and secondly, to unlock locked interfaces.

### SUMMARY

It is an objective of the present disclosure to provide a portable medical infusion pump with a user interface providing simplified user interaction. It is a further object to provide a method for unlocking the user interface of a portable medical infusion pump by a few simple steps while maintaining protection from inadvertent use.

The objectives are achieved by the medical infusion pump and the method to unlock a portable medical infusion pump as disclosed herein.

In an embodiment, a portable medical infusion pump configured to deliver fluid medicament to a using person. The medical infusion pump may be an ambulatory infusion pump. For example, the infusion pump may be an insulin pump similar to the YpsoPump marketed by Ypsomed in many European countries. The medical infusion pump according to implementations includes a user interface at least including a depressible button and a touch sensitive display.

The touch sensitive display may be a combination of a transparent touch sensitive layer and an LCD or OLED display capable of displaying background, characters or symbols in black and white, a single color in varying shades or in full color. Further, the portable medical infusion pump includes an electronic controlling assembly. The electronic controlling assembly includes a microcontroller or processor configured to execute instruction or programs stored in a part of the electronic controlling assembly, for example, a non-transitory computer readable medium or memory. The electronic controlling assembly may be considered a small and lean computer. The electronic controlling assembly is functionally coupled to the user interface, including the button and the touch sensitive display. The electronic controlling assembly is configured to create and display screens on the touch sensitive display as well receive signals from the same. Moreover, the electronic controlling assembly is configured to receive signals from the button. The microcontroller or processor is configured to process received signals, and in response, create and display a screen on the touch sensitive display.

The depressible button is to be construed in its broadest sense as a push button where depressing the button is an actual geometrically detectable movement of at least a part of the button or, on the other hand, button depression may be a type of touch or pressure detection, where no movement is visible. Important to this context is that the button is functionally coupled to the controlling assembly, capable of signaling a button depression and a duration of the depression. One depression event is a continuous depression (as the button is a push button and not a switch).

As a using person operates the portable medical infusion pump through the user interface with the touch sensitive display, the electronic controlling assembly is further configured to prevent inadvertent operation of the medical infusion pump by putting the user interface into a locked mode (or state) when the using person is not interacting with the user interface on purpose. In the locked mode only restricted interaction through the user interface is possible. The medical infusion pump is required to undergo an unlock process when the using person intentionally wants to interact with the touch sensitive display. In a first step the touch sensitive display is activated and optionally a status screen is displayed on the touch sensitive display. This is accomplished through depression of the button for an at least predefined amount of time. The predefined amount of time may vary between less than a second up to several seconds. The electronic control assembly detects the depression as well as the duration of the depression. As a consequence of correct depression of the button for at least the predefined amount time, the electronic control assembly activates the touch sensitive screen and optionally creates and displays the status screen, e.g., including information about current or past medication delivery or filling level of a drug reservoir present in the medical infusion pump. In order to proceed with the unlocking process, the using person is required to touch the touch sensitive screen with a finger and perform a swipe movement with the finger on or across the touch screen. When receiving the according signal generated by the swiping movement at the electronic controlling assembly, the electronic controlling assembly creates and displays a first screen on the touch sensitive display.

Optionally, if the time span between depression of the button and the swipe movement exceeds a predetermined period, the electronic controlling assembly may abort the unlocking procedure and deactivate the touch sensitive screen again. The first screen comprises at least two graphical elements with one of the elements graphically emphasized or highlighted. The emphasis on one of the graphical elements is intended to attract the attention of the using person and guide the person to touch the emphasized graphical element with a finger. When the user touches the emphasized graphical element with a finger, the electronic controlling assembly receives the according signal and, as a consequence, creates and displays an updated screen. The updated screen shows the same number of graphical elements. However, the emphasized and subsequently touched graphical element is changed to a non-emphasized state and also changed graphically with the aim to graphically represent confirmation of correctly touching the emphasized graphical element (confirmed state). The changed graphical element may be referred to herein as a graphical confirmation element. Moreover, one of the not yet emphasized graphical elements has become emphasized on the updated screen, and hence attracting the using person's attention. Upon touching the newly emphasized graphical element the electronic controlling assembly creates and displays another updated screen on which the newly emphasized graphical element is de-emphasized and changed to the confirmed state represented by a graphical confirmation element. Moreover, yet another of the not-yet touched elements becomes emphasized. This process of the touching and changing graphical elements is repeated until all graphical elements have reached the confirmed state. Once all graphical elements have been changed into graphical confirmation elements, the electronic controlling assembly fully unlocks the user interface for further interaction of the using person with the medical infusion pump. Optionally, a user menu is created and displayed by the electronic controlling assembly on the touch sensitive display upon completion of the unlocking process. In a variant, a status screen is created and shown upon completion of the unlock process and the mentioned user menu may be reached by another swipe movement with a finger over the touch sensitive display. It has to be mentioned that the start of the unlock process is not compulsorily done by depressing the button and the subsequent touching swipe movement, and other starting sequences are possible without divergence from the present disclosure. In other words, the user guidance sequence at the touch display (to ascertain that the user is determined and prepared to access the device functions) may be triggered in many suitable ways. For example, the first screen with the graphical elements may be triggered just by depressing the button in a predefined sequence of depression events, without further swiping movements at the display. Alternatively, if the pump is equipped with appropriate (electronic) sensors functionally coupled to the electronic controlling assembly, the display of the first screen may be triggered by, e.g., shaking the pump, if the sensors would be at least one acceleration sensor or the like.

If the using person does not complete the unlock process for any reason or within a predefined amount of time, optionally, a time-out function may be applied by the controlling assembly, the time function leading to the abortion of the unlock process.

In alternative embodiments of the unlock process described above, not all graphical elements which are to be touched may be shown initially on the first screen, and vice versa not all graphical elements may stay on the following screens after being touched, e.g., one or more new elements may appear on the second and further screens and touched elements may disappear after being touched. In one example, touched and emphasized graphical elements will not be changed to a non-emphasized graphical element on the following screen, but will completely disappear. If on the first screen all to-be-touched graphical element are shown, with each touching of an emphasized graphical, one more graphical element will have disappeared on the following screen, until all graphical elements have disappeared, and the user interface of the pump is set into the unlocked mode. In another example, the first screen does not show all to-be-pressed graphical elements. Instead, only a fraction of the graphical elements, e.g., one graphical element is shown. After touching this graphical element, this graphical element is rendered in its non-emphasized form, and another graphical element appears in an emphasized mode, which indicates that the other graphical element shall be touched as well. This process will be repeated until the unlocked state of the user interface of the pump is reached. A combination of the preceding examples is also possible, where only one graphical element is shown on each screen.

In one aspect, the graphical elements as described above do not contain alphanumeric characters. This has the advantage that illiterate people or small children do not get confused by numbers or letters possibly indicating something they don't understand.

In one aspect, the emphasis of the emphasized graphical elements is the result of an increased luminosity or brightness of the emphasized graphical element on the touch sensitive display. Alternatively, the emphasis may also be added by changing the color for the emphasized graphical element. In an even further alternative, the emphasis may be added by animating the emphasized graphical element, e.g., by wiggling, blinking or rotating the emphasized graphical element. Combinations of the alternatives are of course also possible. Moreover, the kind and degree of desired emphasis may be selectable and adjustable in the user menu of the medical infusion pump in a further variant. In yet further alternatives or combinations of the above-mentioned alternatives, emphasis may be added by changing size or shape of the graphical element to be emphasized. The graphical elements may be arranged in variable orientations and arrangements on the touch sensitive display, e.g., horizontally, vertically, or diagonally distributed across the touch sensitive display. The order in which the graphical elements become highlighted may be varied automatically or set manually.

In one aspect, the graphical elements as described above (prior to changing into the confirmed state) consist of simple geometrical shapes. Optionally, frames surround each of the elements. Alternatively and optionally, one frame surrounds all graphical elements. The simple geometrical shape may be a square, a rectangle, a triangle or any other simple polygon. Preferably but optionally, the simple geometrical shape may be filled on the display to increase readability.

In one further possible aspect, the emphasized graphical element is changed into the graphical confirmation element as soon as the using person touches the graphical emphasized element with a finger. In this aspect, the electronic controlling assembly just changes the emphasized graphical element into the confirmation element when receiving a touch signal from the touch sensitive display. Upon removing the finger from the touch sensitive display, the full new screen is generated and displayed on the touch sensitive display. This aspect has the advantage that a using person is getting positive feedback earlier.

In one aspect, the graphical confirmation elements include a single symbol describing confirmation or completion of an action in a figurative way, so it may be understood by the broadest possible range of users. The single symbol may include a check mark. Alternatively, the symbol may include a happy emoji or emoticon signaling confirmation. The emoji or emoticon may include a smiley or a thumbs-up symbol.

One aspect relates to operation of the medical infusion pump when in locked mode. When in locked mode, a using person may only perform limited variety of operations through the user interface. In one alternative, a using person may be able to program a so-called blind bolus. If the medical infusion pump is a conventional insulin pump with an infusion set this means that a using person is not required to interact with the display to program a bolus. For example, the pump may be kept in a trouser pocket while programming the bolus. In the present aspect a using person may program a blind bolus with the user interface being in locked mode by repeatedly depressing the button. As described above, the button is also used to start the unlock procedure. In order to discriminate the different commands of starting the unlock procedure and programming a blind bolus, the blind bolus programming may be started by depressing the button using a double- or triple-click-like depressing sequence. The blind bolus programming procedure is started if the electronic controlling assembly receives signals according to such a sequence. In order to program the actual blind bolus, the using person presses the button again for a number of times corresponding to a desired size of the blind bolus. Optionally, the electronic controlling assembly listens for button depressing signals for a predefined time span, e.g., for 20 seconds. As mentioned, the number of depression events corresponds to a size of the bolus, in some embodiments the size of the bolus scales linearly with the number of depression events. In case the medicament to be administered is insulin, one depression event may correspond to one I.U. (International Unit) of insulin. Alternatively, the amount per depression may correspond to less or more than one I.U. In other alternatives with e.g., other drugs, each number of depressions may correspond to a predefined amount of drug. For example, two depressions may correspond to 10 mg of drug and three depressions to 20 mg of drug. After the desired number of depressions is executed by the using person, the electronic controlling assembly operates the infusion pump to administer the programed bolus.

One embodiment relates to a method to unlock the user interface of a portable medical infusion pump from a locked state, where only restricted interaction with a user is possible to an unlocked state where a user may fully operate and control the medical infusion pump. The medical infusion pump in this aspect corresponds widely to the medical infusion pump above. The (graphical) elements of the medical infusion pump in this aspect correspond to the element described above. The electronic controlling assembly of the medical infusion pump is configured to perform the steps in interaction with a using person, to unlock the user interface as follows. The electronic controlling assembly starts the unlock procedure after the button is depressed for at least a predefined amount of time. This activates the touch sensitive display of the user interface. Then, after a swipe movement of a finger of the user is detected on the touch sensitive display, the electronic controlling assembly creates and displays a first screen on the touch sensitive display, the first screen displaying at least two graphical elements, wherein one of the graphical elements is graphically emphasized relative to the other graphical elements. Subsequently, after a touch of the graphically emphasized element is detected on the touch sensitive display, the electronic controlling assembly creates and displays an updated screen on the touch sensitive display. On the updated screen, the emphasized element is changed to a graphical confirmation element representing confirmation of the touching and further changing another of the at least two graphical elements to be graphically emphasized. The updating of the screen with further detected touches of graphically emphasized elements is repeated until all graphical elements have been changed to represent graphical confirmation elements and finally followed by the full unlocking of the user interface.

The method may be amended in an alternative. In the alternative activation of the touch sensitive screen and before receiving the signal of the swipe movement, the electronic controlling assembly may create and display a status screen. The status screen may display information about past medicament or drug administration, information about the filling level of a medicament or drug reservoir present in the portable medical infusion pump. It further may comprise information about time and date and/or error message, battery status or any other information considered helpful (like received blood glucose measurement levels). It should be noted that the status screen may not only be shown in the event of the unlocking process, it may also make sense to show the status screen at other occasions. And the status screen may also be called in the menu of the portable medical infusion pump after the unlocking process is completed.

The method may be amended in an alternative. The described swipe movement may consist of a movement of a single finger of the user continuously touching the touch sensitive display from approximately a left edge of the touch sensitive display to approximately a right edge of the touch sensitive display. Alternatively, the swipe movement may be adapted to be from the right edge to the left edge, or from bottom edge to top edge, or from top edge to bottom edge. The swipe movement may be along a diagonal of the display. In even a further alternative, the touch sensitive display may be a so-called multi-touch display, capable of detecting touches of multiple fingers at the same time, which gives more alternatives to the swiping movement without departing from the present disclosure.

The method may be amended in an alternative such that the medical infusion pump further comprises an acoustic emitter, such as buzzer or a speaker, controlled by the electronic controlling assembly. The electronic controlling assembly may be configured to create and emit one or more acoustic signals through the acoustic emitter during and/or after unlocking the user interface. In a further variant of the method, the predefined amount of time the button may have to be pressed continuously to start the unlock process is at least one second or more. In alternatives, the button is to be pressed between one and three seconds, or two and up to six seconds.

The method may be further and possibly altered such that the electronic controlling assembly is stopping the time needed for completing the unlock procedure. For example, if the described unlock procedure is not completed within a predefined duration limit, the electronic controlling assembly quits the unlock procedure and sets back the user interface into the locked state. The duration limit may be settable by the using person or a health care professional. A duration limit may be set during manufacturing of the portable medical infusion pump. This duration limit may be 30 second or shorter. In one alternative it may be 15 seconds or shorter. Optionally it may set to a value of 10 seconds.

### BRIEF DESCRIPTION OF THE DRAWINGS

The disclosed subject matter will be explained in more detail in the following text with reference to preferred exemplary embodiments, which are illustrated in the attached drawings, in which:

FIG. 1 depicts the portable medical infusion pump in a perspective view with the touch sensitive display switched off.

FIG. 2 depicts the portable medical infusion pump with the touch sensitive display switched on but with a user interface in a locked mode showing a lock symbol on the status screen.

FIGS. 3a-3e depict the portable medical infusion pump at different states of the unlock process according to an embodiment of the present disclosure.

FIG. 4 depicts a portable medical infusion pump during an alternative embodiment of the present disclosure.

The reference symbols used in the drawings, and their primary meanings, are listed in summary form in the list of designations. In principle, identical parts are provided with the same reference symbols in the figures.

### DETAILED DESCRIPTION

### Definitions

In the present context, the terms “substance”, “drug”, “medicament” and “medication” are to be understood to include any flowable medical formulation suitable for controlled administration through a means such as, for example, a cannula or a hollow needle, and comprises a liquid, a solution, a gel or a fine suspension containing one or more medical active ingredients. A medicament can be a composition comprising a single active ingredient or a pre-mixed or co-formulated composition with more than one active ingredient present in a single container. Medication includes drugs such as peptides (e.g., insulin, insulin-containing drugs, GLP-1 containing drugs or derived or analogous preparations), proteins and hormones, active ingredients derived from, or harvested by, biological sources, active ingredients based on hormones or genes, nutritional formulations, enzymes and other substances in both solid (suspended) or liquid form but also polysaccharides, vaccines, DNA, RNA, oligonucleotides, antibodies or parts of antibodies but also appropriate basic, auxiliary and carrier substances.

The term “distal” is meant to refer to the direction or the end of the drug delivery device carrying an injection needle or an injection cannula, whereas the term “proximal” is meant to refer to the opposite direction or end pointing away from the needle or cannula.

The term “injection system” or “injector” refers to a device that is removed from the injection site after each medication event or drug delivery process, whereas the term “infusion system” refers to a device with a cannula or needle that remains in the skin of the patient for a prolonged period of time, for example, several hours.

The term “medical infusion pump” preferably refers to portable medical infusion pumps.

The term “using person” refers to a potential patient wearing and using device, but it refers to person assisting a patient (like a health care professional) and hence just operating a device.

### DETAILED DESCRIPTION OF THE FIGURES

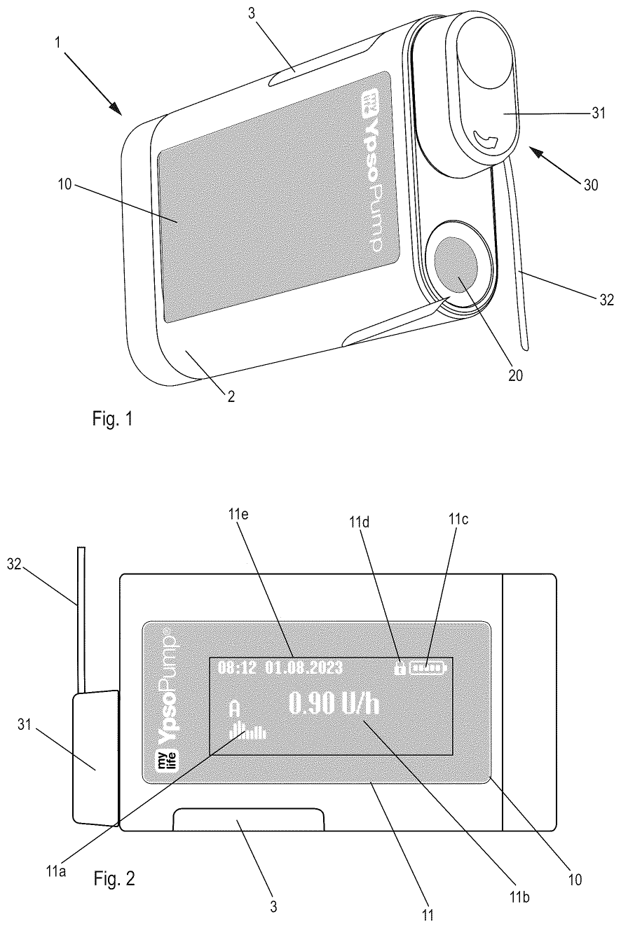

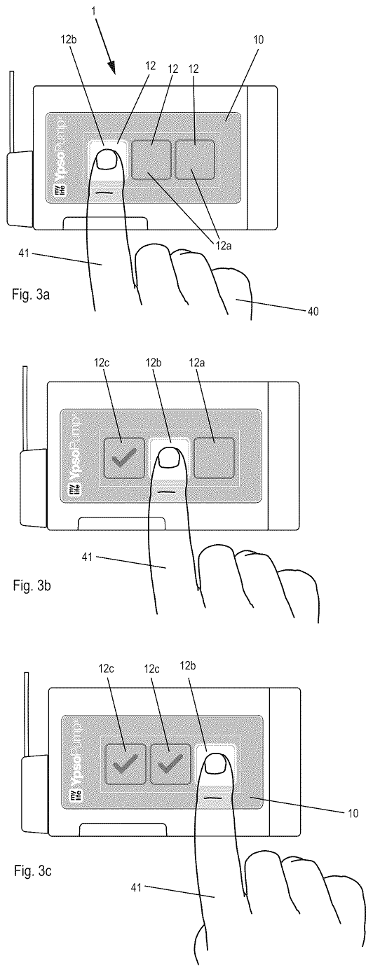

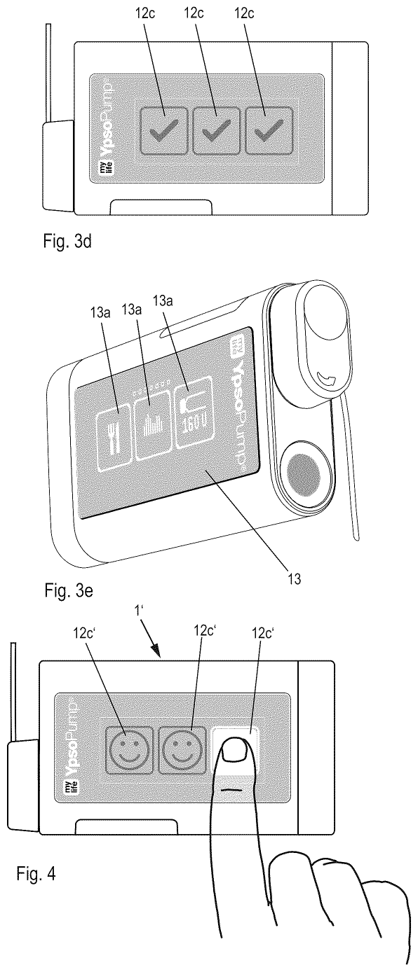

FIG. 1 shows a perspective view of the portable medical infusion pump 1. Pump 1 is in this example an insulin pump of the type YpsoPump manufactured by Ypsomed. However, other types of pumps do not deviate from the present disclosure. The portable medical infusion pump 1 according to the present disclosure comprises a user interface which comprises in the shown example of button 20 and the touch sensitive display 10. More components for the user interface are of course possible. The pump 1 further comprises a housing 2 with a window 3 which allows for a simple check of an inserted medicament cartridge (not shown), e.g., an insulin cartridge. Attached to the pump 1 is the infusion set 30, of which the infusion set adapter 31 and infusion tubing 32 are shown. The infusion set adapter 31 is coupled to the housing 2 of the pump 1 and establishes a fluid path between the inserted medicament cartridge (not shown) and the infusion tubing 32. In FIG. 1, the pump 1 is shown in a state where the touch sensitive display is switched off and the user interface is in its locked state. As provided herein, the user interface may be put in a locked state and in an unlocked state by the controlling assembly (not shown). In order to change the state from the locked into the unlocked state a using person is required to interact with the user interface and perform an unlock process. The unlock process is started by the using person depressing button 20 with a finger 41 of a hand 40 of the using person (hand 40 and finger 41 are shown e.g., on FIG. 3a), preferably for at least predefined duration. When the control assembly receives the signal from button 20 indicative of the start of the unlock process, it activates the touch sensitive display 10. Preferably (but not necessarily) the touch sensitive display 10 displays a status screen 11 as shown in FIG. 2. The status screen 11 comprises several elements to inform the using person about the current status of the portable medical infusion pump 1. The number and kind of elements shown may vary without deviation from the inventive idea. The status screen 11 in this example shows the currently running basal medicament profile 11a (e.g., basal insulin profile), the current infusion rate, 11b shown as insulin unit per hour (U/h), the battery status 11c, a lock symbol 11d (in case of locked user interface), and time and date 11e. In this state, the using person may proceed with the unlock process by swiping over the touch sensitive display 10 with a finger 41 from e.g., the left to the right end of the touch sensitive display 10. When receiving the swiping movement signal, the control assembly of the portable medical infusion pump 1 creates and displays the screen as shown in FIG. 3a. The touch sensitive display 10 displays several graphical elements 12, in this example, three graphical elements 12. One of the graphical elements 12 is a graphically emphasized element 12b and the other two are non-emphasized graphical elements 12a. The graphical emphasis in FIG. 3a is added by filling the square of the graphical element and increasing the brightness of the graphical element. By this, attention is drawn to the graphically emphasized element 12b motivating the using person to touch the graphically emphasized element 12b with his or her finger 41. As the controlling assembly receives a signal from the touch sensitive display 10, which signals the touch of the graphically emphasized element 12b, it creates and displays another screen, which is shown in FIG. 3b. This screen still shows three graphical elements 12, however, the most left one, which used to be shown as graphically emphasized element 12b, is changed into a graphical confirmation element 12c comprising a tick in the shown square signaling even illiterate users the completion of the process step. In addition, the graphical element in the middle of the screen on FIG. 3b is changed as well. Instead of a graphical element 12a (FIG. 3a) an emphasized graphical element 12b is shown, again, attracting the attention of the using person. The third graphical element is still shown as graphical element 12a. If then the using person touches the newly emphasized element 12b, the control assembly receives the signal indicative of this touching action and displays a further screen as shown in FIG. 3c. Analogously, as with the change from FIG. 3a to FIG. 3b, the graphically emphasized element 12b is changed into a graphical confirmation element 12c. Moreover, the next element (right side of the touch sensitive display 10) is changed into graphically emphasized element 12b. After the third graphically emphasized element 12b (FIG. 3c) is touched by the using person, the unlock process is completed. The control assembly may then unlock the user interface and display the menu 13 of the portable medical infusion pump 1 as shown in FIG. 3e or optionally display the screen as shown in FIG. 3d where all graphical elements 12 are changed into graphical confirmation elements 12c symbolizing completion of the unlock process. The screen of FIG. 3d may be displayed for a pre-defined amount of time, e.g., one or two seconds, before the screen of FIG. 3e is shown, which displays the pump menu 13 comprising the menu items 13a. If, during the unlock process, the using person touches for example a non-emphasized graphical element 12a or a graphical confirmation element 12c, the control assembly may detect an according signal but does not create and/or display a changed screen as it waits for a correct touch activity. Moreover, if a started unlock process is not completed by the using person within a pre-defined time window, the control assembly may switch off the touch sensitive display again and hence, cancel the unlock process. Furthermore, it may be possible to cancel the unlock process by the user. This may be possible by the user depressing button 20 after the touch sensitive display 10 was activated by the control assembly.

FIG. 4 shows an alternative, with the difference between FIGS. 3a to 3d and the alternative of FIG. 4 is the differing graphical confirmation element 12c′. The portable medical infusion pump 1′ is very similar to the portable medical infusion pump 1. The difference may be limited to changes in the firmware installed on the control assembly. As can be seen in FIG. 4, the graphical confirmation element 12c′ comprises a smiley-symbol instead of the tick in graphical confirmation element 12c. The unlock process itself may be identical to the one described for FIGS. 3a to 3e.

While the implementations have been described in detail in the drawings and foregoing description, such description is to be considered illustrative or exemplary and not restrictive. Variations to the disclosed embodiments can be understood and effected by those skilled in the art and practicing the claimed invention, from a study of the drawings, the disclosure, and the appended claims. In the claims, the word “comprising” does not exclude other elements or steps, and the indefinite article “a” or “an” does not exclude a plurality. The mere fact that certain elements or steps are recited in distinct claims does not indicate that a combination of these elements or steps cannot be used to advantage, specifically, in addition to the actual claim dependency, any further meaningful claim combination shall be considered disclosed.

### LIST OF DESIGNATIONS

1 Portable medical infusion pump
Portable medical of alternative embodiment
2 Housing
3 Window
10 Touch sensitive display
11 Status screen
11a Basal program
11b Infusion rate
11c Battery level
11d Lock symbol
11e Time and date
12 Graphical elements (group)
12a (Single) graphical element
12b Graphically emphasized element
12b′ Graphically emphasized element
12c Graphical confirmation element
12c′ Alternative graphical confirmation element
13 Menu
13a Menu item(s)
20 Button
30 Infusion set
31 Infusion set adaptor
32 Infusion tubing
40 hand (of using person)
41 Finger

What is claimed is:

1. A portable medical infusion pump configured for infusing a fluid drug, the medical infusion pump comprising:
a user interface comprising a touch sensitive display configured for user interaction with the medical infusion pump and a button separate from the touch sensitive display; and
an electronic controlling assembly comprising a microcontroller or a processor configured for operating and controlling the medical infusion pump,
wherein the electronic controlling assembly is configured to place the user interface into a locked state and into an unlocked state,
wherein in the unlocked state, the infusion pump is configured for interaction by a user through the user interface, and in the locked state, the infusion pump is configured for relatively restricted interaction by the user through the user interface,
wherein the button is adapted to start an unlock process for the user interface, the unlock process comprising at least the following steps:
pressing the button for at least a predefined amount of time by the user,
activation of the touch sensitive display of the user interface by the electronic controlling assembly in response to a signal received by the electronic controlling assembly from the button,
receiving, at the electronic controlling assembly, a signal associated with a swiping of a finger of the user over the touch sensitive display,
generating and displaying, by the electronic controlling assembly, a first screen on the touch sensitive display after receiving the signal associated with the swiping, the first screen displaying at least two graphical elements, wherein one of the at least two graphical elements is graphically emphasized relative to another of the at least two graphical elements,
receiving a signal at the electronic controlling assembly associated with touching the graphically emphasized element with the finger of the user,
after receiving, by the electronic controlling assembly, the signal associated with touching a screen area of the graphically emphasized element, the electronic controlling assembly generates and displays an updated screen with the graphically emphasized element changed to a graphical confirmation element representing confirmation of the touching and further changing another of the at least two graphical elements to being graphically emphasized,
repeating the steps of touching and updating the screens until all of the at least two graphical elements have been changed to represent graphical confirmation elements, and
finalizing the unlock process by unlocking the user interface.

2. The portable medical infusion pump of claim 1, wherein the graphical elements do not contain alphanumerical characters.

3. The portable medical infusion pump of claim 1, wherein the graphical emphasis for the graphically emphasized element comprises increasing a luminosity or changing a size, a shape or a color of the graphically emphasized element.

4. The portable medical infusion pump of claim 1, wherein the graphical emphasis for the graphically emphasized element comprises animating the graphically emphasized element.

5. The portable medical infusion pump of claim 1, wherein the graphical elements, prior to changing to graphical confirmation elements, consist of a simple geometrical and filled shape.

6. The portable medical infusion pump of claim 1, wherein the graphical confirmation elements comprise a check mark, a smiley, or a thumbs-up-symbol.

7. The portable medical infusion pump of claim 1, wherein in the locked state, the portable medical infusion pump is configured for the user to program a blind bolus of the fluid drug, the blind bolus being programmed by repeatedly pressing the button in predefined sequences.

8. The portable medical infusion pump of claim 7, wherein a number of button pressing events in the predefined sequences scales with a size of a bolus to be programmed.

9. A method of unlocking a user interface of a portable medical infusion pump from a locked state, where only a restricted interaction by a user is possible, to an unlocked state, where the portable medical infusion pump is configured to be fully operated and controlled by the user, the medical infusion pump comprising:
a user interface comprising a touch sensitive display and a button separate from the touch sensitive display allowing for user interaction with the medical infusion pump; and
an electronic controlling assembly at least comprising a microcontroller or a processor configured for operating and controlling the medical infusion pump, the electronic controlling assembly configured to receive signals from the button and the touch sensitive display and to generate and display screens on the touch sensitive display,
wherein the electronic controlling assembly is configured to perform at least the following steps in interaction with the user to unlock the user interface:
starting an unlock procedure after the button is pressed for at least a predefined amount of time,
activating the touch sensitive display of the user interface,
generating and displaying a first screen on the touch sensitive display after receiving a signal at the electronic controlling assembly associated with a swipe movement of a finger of the user detected on the touch sensitive display, the first screen displaying at least two graphical elements, wherein one of the at least two graphical elements is graphically emphasized relative to another of the at least two graphical elements,
generating and displaying an updated screen on the touch sensitive display after receiving a signal associated with a touch of the graphically emphasized element detected on the touch sensitive display, wherein on the updated screen, the graphically emphasized element is changed to a graphical confirmation element representing confirmation of the touching and further changing the another of the at least two graphical elements to be graphically emphasized,
repeating updating of the screen based on further received signals associated with detected touches of graphically emphasized elements until all of the at least two graphical elements have been changed to graphical confirmation elements, and
unlocking of the user interface.

10. The method of claim 9, wherein the electronic controlling assembly is further configured to generate and display a status screen on the touch sensitive display after activating the touch sensitive display but prior to receiving the signal at the electronic controlling assembly associated with detecting the swipe movement, and prior to generating the displaying the first screen.

11. The method of claim 9, wherein the swipe movement comprises a movement of a single finger of the user continuously touching the touch sensitive display from approximately a left edge of the touch sensitive display to approximately a right edge of the touch sensitive display.

12. The method of claim 9, wherein the medical infusion pump further comprises an acoustic emitter controlled by the electronic controlling assembly, and wherein the electronic controlling assembly is configured to generate and emit an acoustic signal through the acoustic emitter during or after unlocking the user interface.

13. The method of claim 9, wherein the button is pressed for at least a predefined amount of time of one second or more.

14. The method of claim 9, wherein the button is pressed for at least a predefined amount of time of two seconds or more.

15. The method of claim 9, wherein if the unlock procedure is not completed within 30 seconds, the electronic controlling assembly is configured to cancel the unlock procedure and to re-lock the portable medical infusion pump in the locked state.
